(19) United States
(12) Patent Application Publication
(10) Pub. No.: US 2004/0063961 A1
(43) Pub. Date: Apr. 1, 2004

(54) Crystalline forms of cerivastatin sodium

(76) Inventors: Paul Adriaan Van Der Schaaf, Allschwil (CH); Fritz Blatter, Reinach (CH); Martin Szelagiewicz, Munchenstein (DE); Nicole End, Oberwil (CH)

Correspondence Address: CIBA SPECIALTY CHEMICALS CORPORATION, PATENT DEPARTMENT, 540 WHITE PLAINS RD, P O BOX 2005, TARRYTOWN, NY, 10591-9005, US

(21) Appl. No.: 10/451,133

(22) PCT Filed: Dec. 12, 2001
(86) PCT No.: PCT/EP01/14602

(30) Foreign Application Priority Data
Dec. 21, 2000 (EP) 00811225.2

(51) Int. Cl. C07D 213/55

(52) U.S. Cl. 546/341

(57) ABSTRACT

Crystalline forms of Cerivastatin sodium were found, referred to hereinafter as polymorphic Forms X, A1, A2, B, C, D1, D3, D4, E and F. Furthermore, the present invention is directed to processes for the preparation of these crystalline forms and pharmaceutical compositions comprising the crystalline forms.

The present invention is directed to crystalline forms of Cerivastatin sodium, processes for their preparation and pharmaceutical compositions comprising these crystalline forms.

The present invention relates to crystalline forms of Cerivastatin sodium. Cerivastatin sodium is known by the chemical name (3R,5S,6E)-7-(4-(4-fluorophenyl)-2,6-diisopropyl-5-(methoxymethyl)pyridin-3-yl)-3,5-dihydroxy-6-heptenoic acid sodium salt. Cerivastatin sodium has the following formula:

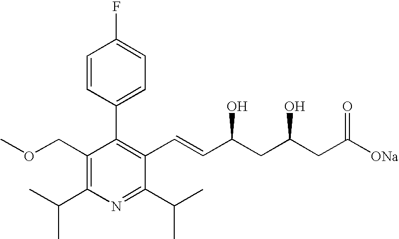

Cerivastatin is a fully synthetic inhibitor of 3-hydroxy-3-methylglutaryl coenzyme-A reductase (HMG-CoA reductase) for the treatment of hyperlipoproteinemia. Processes for the preparation of Cerivastatin sodium are described in EP-A-491,226 and EP-A-617,019. The sodium salt is desirable since it enables Cerivastatin to be conveniently formulated in, for example, tablets, capsules, powders and the like. There is still a need to produce Cerivastatin in a reproducible, pure and crystalline form to enable formulations to meet exacting pharmaceutical requirements and specifications. Furthermore, it is economically desirable that the product is stable for extended periods of time without the need for specialized storage conditions. In the processes in the above mentioned publications Cerivastatin sodium is isolated as a pure compound by lyophilization of an aqueous solution. By these processes Cerivastatin is obtained as a white to off-white amorphous powder. Surprisingly, according to the present invention controlled lyophilization of a concentrated aqueous solution of cerivastatin sodium gave a microcrystalline powder having an X-ray diffraction pattern with only one signal at 2θ=3.30° (i.e. at a d-value of 26.7 Å). This solid-state form is herein designated as Form X. More surprisingly, further investigations led to the discovery of several other novel crystalline forms of Cerivastatin sodium, herein designated as Form A1, A2, B, C, D1, D2, D3, D4, E and F. The novel crystalline forms of th present invention have a good thermal stability and/or good solubility characteristics.

Accordingly, the present invention is directed to Form X and the following polymorphic Forms A1, A2, B, C, D1, D2, D3, D4, E and F of Cerivastatin sodium:

A crystalline polymorph of (3R,5S,6E)-7-(4-(4-fluorophenyl)-2,6-diisopropyl-5-(methoxymethyl)pyridin-3-yl)-3,5-dihydroxy-6-heptenoic acid sodium salt which exhibits a characteristic X-ray powder diffraction pattern with characteristic peaks expressed in d-values (Å) at

27.9 (vs), 20.8 (m), 15.8 (w), 12.4 (w), 10.4 (w), 9.0 (s), 8.3 (m), 7.1 (vw), 6.7 (vw), 6.1 (w), 5.53 (w), 5.24 (w), 4.86 (w), 4.70 (w), 4.49 (m), 4.18 (vw), 4.11 (w), 3.76 (w), 3.66 (vw);

herein designated as Form A1. Here and in the following the abbreviations in brackets mean: (vs)=very strong intensity; (s)=strong intensity; (m)=medium intensity; (w)=weak intensity; and (vw)=very weak intensity.

A crystalline polymorph of (3R,5S,6E)-7-(4-(4-fluorophenyl)-2,6-diisopropyl-5-(methoxymethyl)pyridin-3-yl)-3,5-dihydroxy-6-heptenoic acid sodium salt which exhibits a characteristic X-ray powder diffraction pattern with characteristic peaks expressed in d-values (Å) at

25.2 (vs), 13.0 (vw), 11.6 (vw), 10.7 (w), 8.9 (s), 7.6 (w), 6.8 (vw), 5.92 (vw), 5.26 (vw), 4.33 (vw), 4.11 (w), 3.85 (w), 3.67 (vw);

herein designated as Form A2.

A crystalline polymorph of (3R,5S,6E)-7-(4-(4-fluorophenyl)-2,6-diisopropyl-5-(methoxymethyl)pyridin-3-yl)-3,5-dihydroxy-6-heptenoic acid sodium salt which exhibits a characteristic X-ray powder diffraction pattern with characteristic peaks expressed in d-values (Å) at

28.0 (vs), 26.1 (m), 20.6 (m), 16.0 (w), 12.2 (w), 10.4 (w), 8.9 (m), 8.4 (w), 8.0 (vw), 7.3 (w), 6.5 (vw), 5.35 (w), 5.17 (vw), 4.73 (vw), 4.38 (vw), 4.13 (vw), 3.80 (vw), 3.73 (w);

herein designated as Form B.

A crystalline polymorph of (3R,5S,6E)-7-(4-(4-fluorophenyl)-2,6-diisopropyl-5-(methoxymethyl)pyridin-3-yl)-3,5-dihydroxy-6-heptenoic acid sodium salt which exhibits a characteristic X-ray powder diffraction pattern with characteristic peaks expressed in d-values (Å) at

29.6 (vs), 14.8 (m), 10.0 (vs), 7.5 (vs), 5.61 (vw), 5.14 (vw), 4.77 (vw), 4.32 (w), 3.76 (vw), 3.38 (vw);

herein designated as Form C.

A crystalline polymorph of (3R,5S,6E)-7-(4-(4-fluorophenyl)-2,6-diisopropyl-5-(methoxymethyl)pyridin-3-yl)-3,5-dihydroxy-6-heptenoic acid sodium salt which exhibits a characteristic X-ray powder diffraction pattern with characteristic peaks expressed in d-values (Å) at

30.3 (s), 15.3 (w), 10.2 (s), 7.7 (m), 7.5 (w), 5.63 (m), 5.13 (s), 4.75 (m), 4.13 (w), 3.76 (w), 3.65 (vw), 3.49 (vw), 3.31 (vw);

herein designated as Form D1.

A crystalline polymorph of (3R,5S,6E)-7-(4-(4-fluorophenyl)-2,6-diisopropyl-5-(methoxymethyl)pyridin-3-yl)-3,5-dihydroxy-6-heptenoic acid sodium salt which exhibits a characteristic X-ray powder diffraction pattern with characteristic peaks expressed in d-values (Å) at

31.4 (s), 15.8 (w), 10.5 (s), 10.1 (w), 9.0 (w), 7.9 (s), 7.5 (w), 7.3 (m), 6.6 (vw), 5.65 (m), 5.19 (s), 4.82 (m), 4.31 (w), 4.18 (vw), 4.08 (vw), 3.85 (vw), 3.76 (vw);

herein designated as Form D2.

A crystalline polymorph of (3R,5S,6E)-7-(4-(4-fluorophenyl)-2,6-diisopropyl-5-(methoxymethyl)pyridin-3-yl)-3,5-dihydroxy-6-heptenoic acid sodium salt which exhibits a characteristic X-ray powder diffraction pattern with characteristic peaks expressed in d-values (Å) at

31.2 (s), 15.7 (w), 10.4 (m), 10.0 (vw), 9.0 (vw), 7.8 (m), 7.5 (w), 7.3 (m), 6.6 (vw), 5.64 (m), 5.14 (s), 4.76 (m), 4.28 (vw), 4.13 (vw), 3.78 (vw), 3.66 (vw);

herein designated as Form D3.

A crystalline polymorph of (3R,5S,6E)-7-(4-(4-fluorophenyl)-2,6-diisopropyl-5-(methoxymethyl)pyridin-3-yl)-3,5-dihydroxy-6-heptenoic acid sodium salt which exhibits a characteristic X-ray powder diffraction pattern with characteristic peaks expressed in d-values (Å) at

29.9 (s), 15.0 (w), 10.0 (s), 8.9 (vw), 7.5 (s), 7.0 (w), 5.64 (m), 5.15 (m), 4.76 (m), 4.40 (vw), 4.29 (vw), 4.09 (vw), 3.74 (vw), 3.62 (vw);

herein designated as Form D4.

A crystalline polymorph of (3R,5S,6E)-7-(4-(4-fluorophenyl)-2,6-diisopropyl-5-(methoxymethyl)pyridin-3-yl)-3,5-dihydroxy-6-heptenoic acid sodium salt which exhibits a characteristic X-ray powder diffraction pattern with characteristic peaks expressed in d-values (Å) at

35.4 (s), 29.9 (m), 25.3 (vs), 17.5 (vw), 12.4 (w), 11.7 (vw), 10.6 (m), 9.4 (w), 8.9 (s), 8.7 (shoulder), 7.5 (m), 7.3 (w), 6.8 (w), 6.2 (m), 5.81 (m), 5.27 (m), 5.13 (w); 4.98 (vw); 4.90 (w); 4.78 (vw); 4.70 (w); 4.63 (vw); 4.56 (w); 4.46 (w); 4.35 (w); 4.16 (s); 4.05 (m); 3.84 (w); 3.76 (m); 3.70 (m); 3.62 (vw); 3.59 (vw); 3.56 (vw); 3.51 (vw); 3.49 (vw); 3.33 (vw); 3.17 (vw); 3.00 (w).

herein designated as Form E.

A crystalline polymorph of (3R,5S,6E)-7-(4-(4-fluorophenyl)-2,6-diisopropyl-5-(methoxymethyl)pyridin-3-yl)-3,5-dihydroxy-6-heptenoic acid sodium salt which exhibits a characteristic X-ray powder diffraction pattern with characteristic peaks expressed in d-values (Å) at

35.0 (m); 27.3 (vs); 21.0 (vw); 11.7 (vw); 11.1 (w); 10.4 (m); 9.1 (s); 8.6 (s); 7.8 (w); 7.0 (vw); 6.6 (vw); 6.04 (m); 5.28 (m); 5.07 (w); 4.82 (w); 4.72 (m); 4.51 (vw); 4.37 (vw); 4.27 (m); 4.11 (m); 3.81 (s); 3.64 (w); 3.57 (vw); 3.51 (vw); 3.43 (vw); 3.36 (vw); 3.25 (w); 2.69 (w)

herein designated as Form F.

A discussion of the theory of X-ray powder diffraction patterns can be found in “X-ray diffraction procedures” by H. P. Klug and L. E. Alexander, J. Wiley, New York (1974).

Furthermore, the present invention is directed to processes for the preparation of Forms X, A1, A2, B, C, D1, D2, D3, D4, E and F.

Form X of Cerivastatin sodium can, for example, be obtained by controlled lyophilisation of an aqueous solution of the amorphous form. Preferably, a concentrated aqueous solution is used. If desired, during the preparation process an additional seeding with Form X can be carried out.

Form A1 of Cerivastatin sodium is generally prepared from Form X or the amorphous form by equilibration of either one or a mixture of these forms under relative humidity conditions ranging from about 30% to 70% relative humidity. A temperature ranging from about 20° C. to 50° C. is preferred. If desired, during the preparation process an additional seeding with Form A1 can be carried out. Form A1 typically contains about 3% of water, which corresponds to about 0.8 mole water per mole of Cerivastatin sodium. Form A1 may contain a slightly variable amount of water.

Form A2 of Cerivastatin sodium is generally prepared from Form X or the amorphous form. For example, the amorphous form or preferably Form X is suspended in a mixture of an organic solvent with water. After heating the suspension to elevated temperature, like 40 to 80° C., the suspension is cooled, for example to about ambient temperature. As organic solvent the use of acetone and acetonitrile and especially a mixture thereof is preferred. If desired, during the preparation process an additional seeding with Form A2 can be carried out.

Form B of Cerivastatin sodium is generally prepared by drying of Form A1 or Form A2 and subsequent treatment at elevated temperatures, like 70 to 150° C. For example, Form B can be prepared by drying of Form A1 or Form A2 and subsequent autoclaving at elevated temperature, like 80 to 130° C. More specifically, Form A1 or Form A2 can be dried under nitrogen at ambient temperature, the dried form is placed a in a closed container with a small residual volume and this sample is autoclaved at 80 to 130° C. (for example, for about 5 to 15 minutes). If desired, during the preparation process an additional seeding with Form B can be carried out. As to the preparation of Form B it is preferred to start from Form A1. Cerivastatin Form B prepared by the above procedure typically contains less than 1% of water.

Form C of Cerivastatin sodium is generally prepared by exposing either Form X or Form A1 or Form A2 to a relative humidity condition of 70 to 100%, especially about 90%. This treatment is preferably carried out for about one to several hours at a temperature range from about 20° C. to 50° C. If desired, during the preparation process an additional seeding with Form C can be carried out.

Forms D1, D2, D3, and D4 of Cerivastatin sodium generally can be produced if either Form X or the amorphous form, or a mixture thereof, is lyophilized in a mixture of an organic solvent, like acetonitrile, and water. Preferably, a 1:1 mixture (by volume) is used. The obtained lyophilized product is treated under a controlled humidity program. Within this program the relative humidity is slightly increased from ambient to 70% relative humidity, then to 90% relative humidity and returning to 70% relative humidity after several hours of exposition to 90% relative humidity. If desired, during the preparation process an additional seeding with the desired form can be carried out.

Form E of Cerivastatin sodium can generally be produced by stirring a suspension of Form A1 in acetone containing a small amount of water.

Form F of Cerivastatin sodium can generally be produced by stirring a suspension of Form A1 in acetone containing a small amount of water and subsequent treatment with acetonitrile.

Another object of the present invention are pharmaceutical compositions comprising an effective amount of crystalline polymorphic Form X, A1, A2, B, C, D1, D2, D3, D4, E and F and a pharmaceutically acceptable carrier.

The polymorphic forms may be used as single components or mixtures.

As to the novel polymorphic forms of Cerivastatin sodium it is preferred that these contain 25-100% by weight, especially 50-100% by weight, of at least one of the novel forms, based on the total amount of Cerivastatin sodium. Preferably, such an amount of the novel polymorphic forms of Cerivastatin is 75-100% by weight, especially 90-100% by weight. Highly preferred is an amount of 95-100% by weight.

The following Examples illustrate the invention in more detail. Temperatures are given in degrees Celsius.

### EXAMPLE 1

Preparation of Polymorphic Form X

Solid Form X of cerivastatin sodium salt is obtained from an aqueous solution of the amorphous form, which is frozen at −80° C. and lyophilis d at 0.05 to 0.1 mbar for 2 to 3 days. Form X is characterized by a single x-ray diffraction peak at 2θ=3.30°, i.e. at a d-value of 26.7 Å (see FIG. 1). Differential scanning calorimetry (DSC) of this solid form reveals the presence of a substantial amount of the amorphous form because a glass transition is observed near 70° C.

### EXAMPLE 2a

Preparation of Polymorphic Form A1

Form A1 of cerivastatin sodium salt is prepared from cerivastatin Form X. 100 mg of crystal Form X are stored over a saturated solution of Mg(NO₃)₂*6H₂O at 43° C. for about 12 hours. The obtained sample is crystalline and corresponds to cerivastatin sodium salt Form A1 (see FIG. 2). A Karl Fischer titration reveals a water content of 3% which indicates the presence of about 0.8 water molecules per mol of cerivastatin sodium salt. Differential scanning calorimetry in a closed sample pan at a heating rate of 20K/min shows a melting point of 124° C. and an enthalpy of fusion of about 41 J/g (see FIG. 6).

### EXAMPLE 2b

Preparation of Polymorphic Form A1

Cerivastatin sodium salt Form X obtained from lyophilization as described in example 1 is stored under controlled change of the relative humidity, i.e. starting from ambient humidity the humidity is slightly raised to 68% at ambient temperature. This relative humidity condition is adequate to initiate the crystallization of cerivastatin Form A1.

### EXAMPLE 3

Preparation of Polymorphic Form A2

Cerivastatin sodium salt Form A2 is prepared by suspending 55 mg of Form X in a mixture of 2.0 ml acetone, 1.0 ml acetonitrile, and 20 μl H₂O, then heating this suspension to 60° C. and keeping the prepared suspension at 60° C. for a few minutes before the suspension is slowly cooled to ambient temperature and stirred at this temperature for three days. The obtained product was filtered and dried in a dessicator over silicagel (see FIG. 2). Form A2 melts at 104° C., with an enthalpy of fusion of about 39 J/g, if measured in a closed DSC sample pan sealed unter normal atmospheric conditions.

### EXAMPLE 4

Preparation of Polymorphic Form B

20 mg of cerivastatin crystal Form A1 are placed in a gold coated stainless steel DSC sample pan and dried for about 16 hours under dry nitrogen at ambient temperature. This sample contains a residual amount of water of about 0.1% to 0.8%. Subsequently the sample is heated, at a rate of 20K/min, to 120° C., and autoclaved at the same temperature for 10 minutes. Obtained is Ceriavstatin Form B and the X-ray diffraction pattern is shown in FIG. 3. DSC in a closed sample pan sealed under dry nitrogen reveals a melting point at 172° C. and an enthalpy of fusion of about 46 J/g (see FIG. 6).

### EXAMPLE 5

Preparation of Polymorphic Form C

Cerivastatin crystal Form C is prepared by equilibrating 100 mg of Form X in a desiccator at a relative humidity condition of 90%, i.e. over a saturated BaCI₂*2H₂O solution at ambient temperature (see FIG. 4). A Karl Fischer titration of the sample taken from the x-ray sample holder at a relative humidity of about 40% shows a water content of about 15.6%.

### EXAMPLE 6

Preparation of Polymorphic Form D1, D2, D3 and D4

Cerivastatin sodium salt Form D1 is prepared from lyophilization of 50 mg of Form X in a mixture of 200 μl acetonitrile and 200 μl of water. The lyophilized product was treated at ambient temperature under a relative humidity which was slightly increased from ambient to 90% and kept there for about 1 to 2 hours. Prolonged exposition of the same sample for additional 15 hours led to Form D2. Subsequently reducing the relative humidity to 70% led to the formation of Form D3, and after another 3 hours at 70% relative humidity Form D4 was obtained (see FIG. 5).

### EXAMPLE 7

Preparation of Polymorphic Form E

120 mg Cerivastatin sodium salt form A1 are suspended in 1.00 ml acetone (Fluka No. 0570) containing 20 μL of water at 20° C. Cerivastatin form E precipitates while stirring at 20° C. The suspension is stirred for 20 hours at the same temperature and then filtrated. Investigation of the obtained solid form by X-ray diffraction shows the diagram of form E as shown in FIG. 7.

### EXAMPLE 8

Preparation of Polymorphic Form F

117 mg of Cerivastatin sodium salt form A1 are suspended in 1.00 ml acetone (Fluka No. 0570) containing 20 μl of water at 20° C. The mixture is stirred for 20 hours at the same temperature and the suspension is filtrated. Pure acetonitrile was used to wash out the residual material from the reaction tube and the residue on the glass filter. Investigation of the obtained solid form by X-ray diffraction shows the diagram of form F as shown in FIG. 8.

### BRIEF DESCRIPTION OF THE DRAWINGS

FIG. 1 is a characteristic X-ray powder diffraction pattern for Form X.

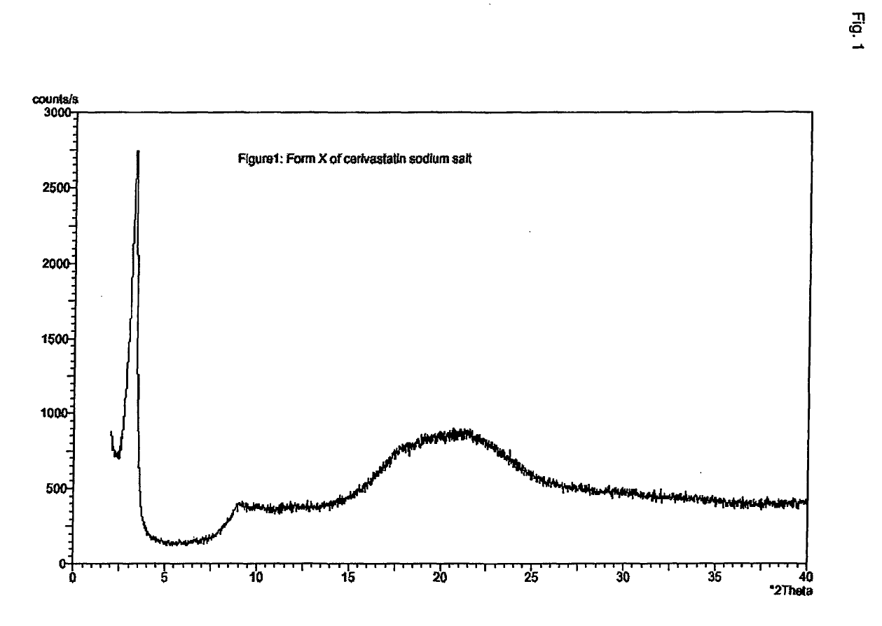

FIG. 2 are characteristic X-ray powder diffraction patterns for Form A1 and Form A2.

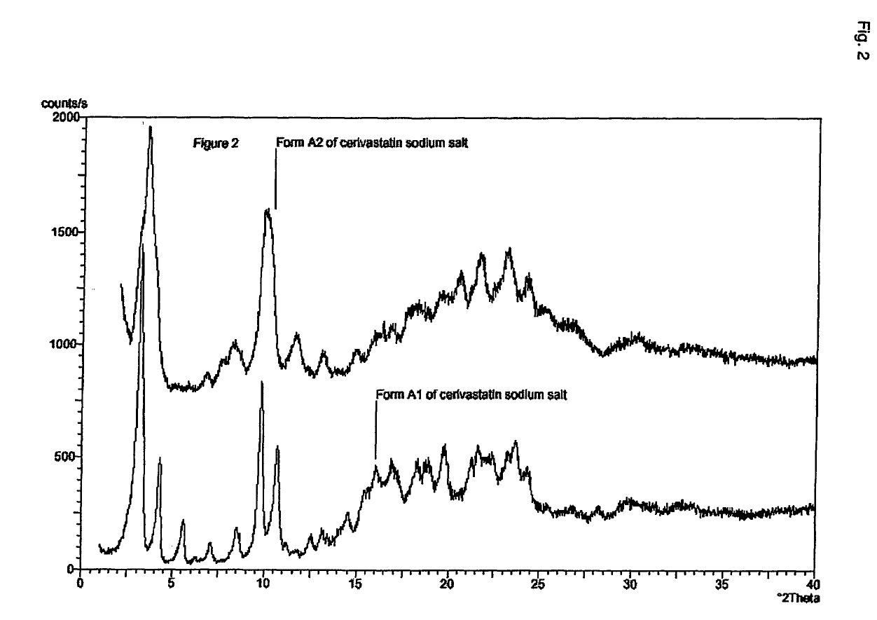

FIG. 3 is a characteristic X-ray powder diffraction pattern for Form B.

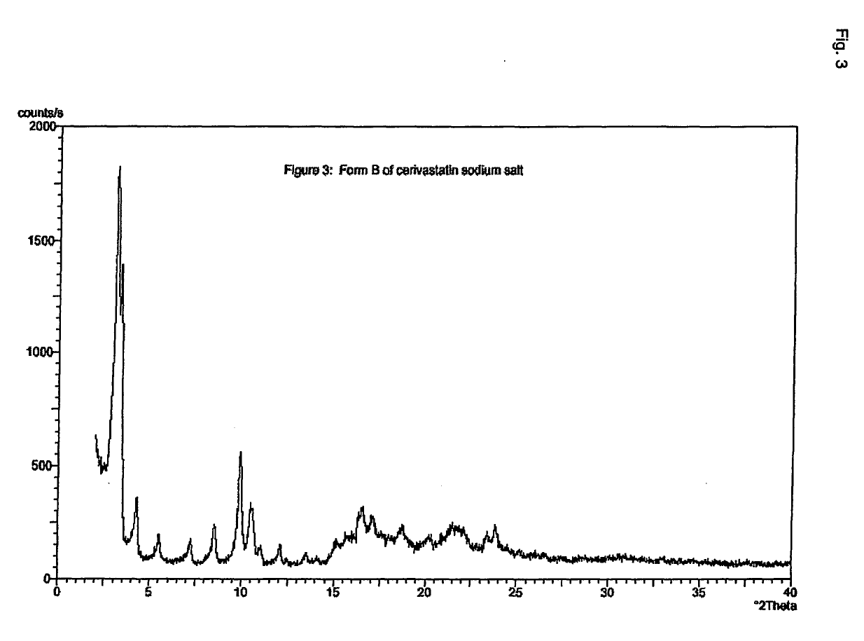

FIG. 4 is a characteristic X-ray powder diffraction pattern for Form C.

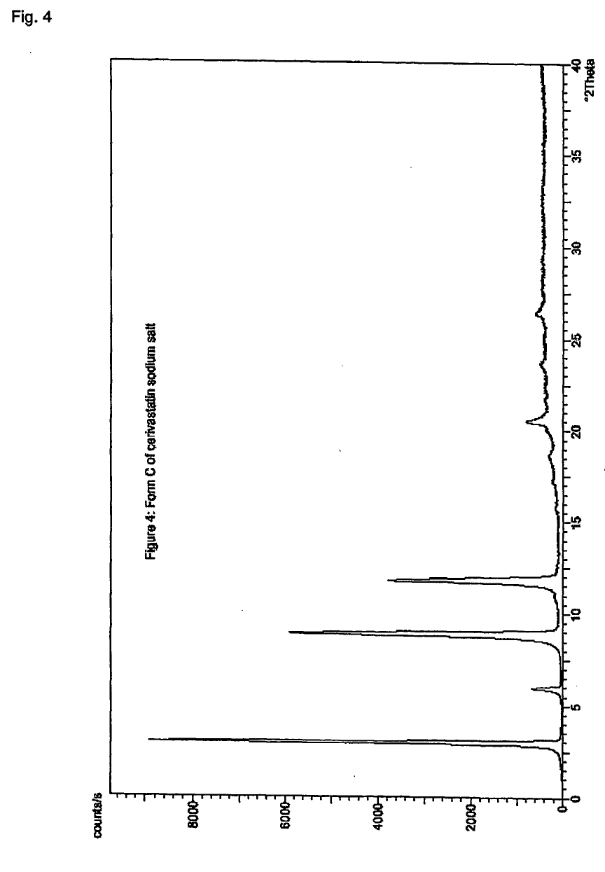

FIG. 5 are characteristic X-ray powder diffraction patterns for Form D1, Form D2, Form D3 and Form D4.

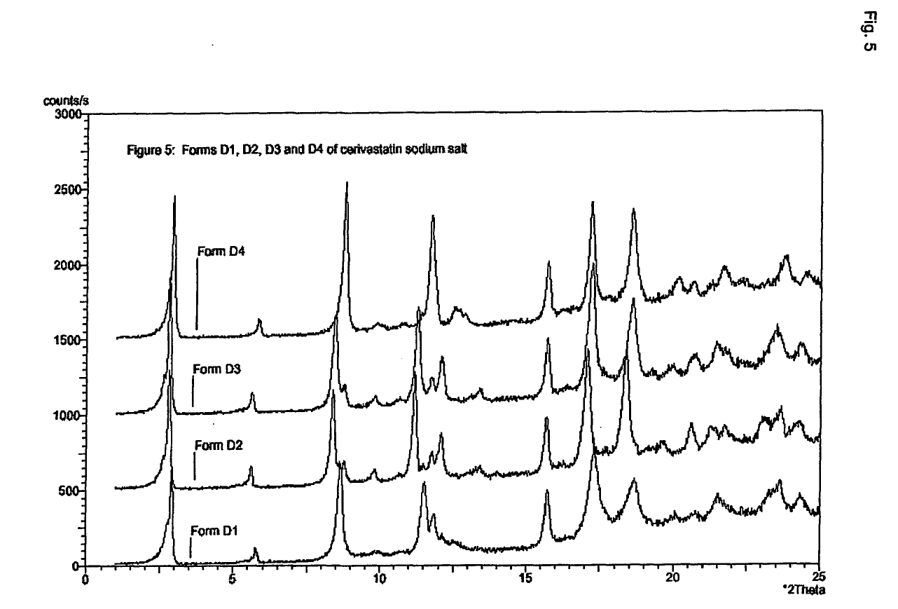

FIG. 6 are characteristic Differential Scanning Calorimetry (DSC) scans of Form A1 and Form B.

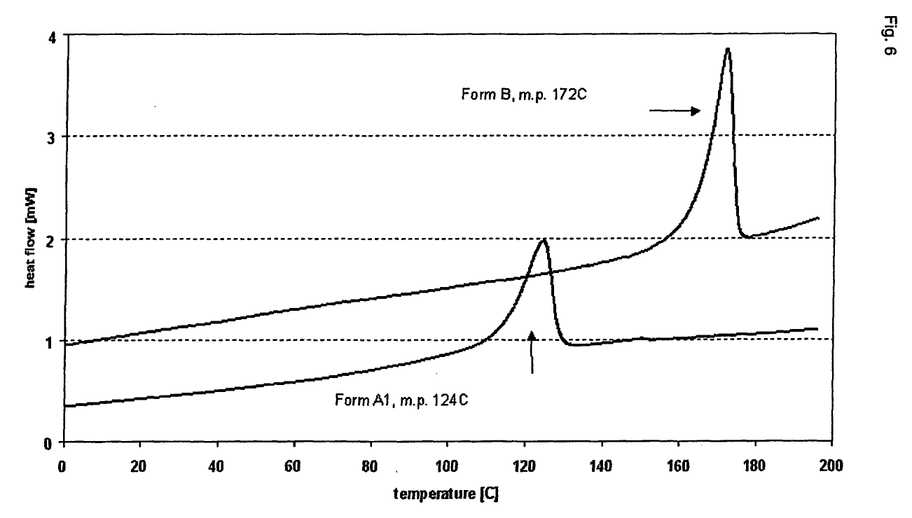

FIG. 7 is a characteristic X-ray powder diffraction pattern for Form E.

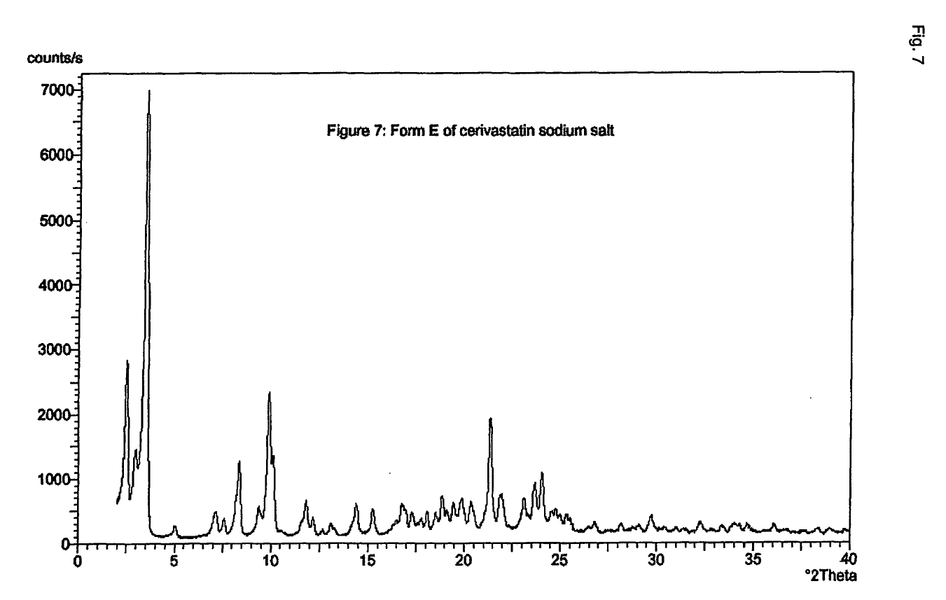

FIG. 8 is a characteristic X-ray powder diffraction pattern for Form F.

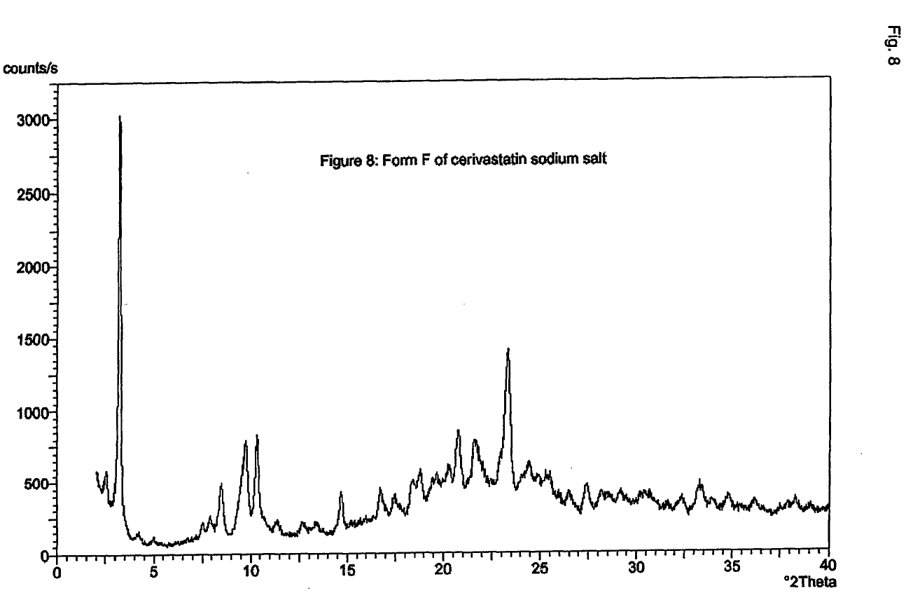

1. A crystalline polymorph of (3R,5S,6E)-7-(4-(4-fluorophenyl)-2,6-diisopropyl-5-(methoxymethyl)pyridin-3-yl)-3,5-dihydroxy-6-heptenoic acid sodium salt which exhibits a characteristic X-ray powder diffraction pattern with characteristic peaks expressed in d-values (Å) at
27.9 (vs), 20.8 (m), 15.8 (w), 12.4 (w), 10.4 (w), 9.0 (s), 8.3 (m), 7.1 (vw), 6.7 (vw), 6.1 (w), 5.53 (w), 5.24 (w), 4.86 (w), 4.70 (w), 4.49 (m), 4.18 (vw), 4.11 (w), 3.76 (w), 3.66 (vw);
wherein (vs)=very strong intensity; (s)=strong intensity; (m)=medium intensity; (w)=weak intensity; and (vw)=very weak intensity.

2. A crystalline polymorph of (3R,5S,6E)-7-(4-(4-fluorophenyl)-2,6-diisopropyl-5-(methoxymethyl)pyridin-3-yl)-3,5-dihydroxy-6-heptenoic acid sodium salt which exhibits a characteristic X-ray powder diffraction pattern with characteristic peaks expressed in d-values (Å) at
25.2 (vs), 13.0 (vw), 11.6 (vw), 10.7 (w), 8.9 (s), 7.6 (w), 6.8 (vw), 5.92 (vw), 5.26 (vw), 4.33 (vw), 4.11 (w), 3.85 (w), 3.67 (vw);
wherein (vs)=very strong intensity; (s)=strong intensity; (w)=weak intensity; and (vw)=very weak intensity.

3. A crystalline polymorph of (3R,5S,6E)-7-(4-(4-fluorophenyl)-2,6-diisopropyl-5-(methoxymethyl)pyridin-3-yl)-3,5-dihydroxy-6-heptenoic acid sodium salt which exhibits a characteristic X-ray powder diffraction pattern with characteristic peaks expressed in d-values (Å) at
28.0 (vs), 26.1 (m), 20.6 (m), 16.0 (w), 12.2 (w), 10.4 (w), 8.9 (m), 8.4 (w), 8.0 (vw), 7.3 (w), 6.5 (vw), 5.35 (w), 5.17 (vw), 4.73 (vw), 4.38 (vw), 4.13 (vw), 3.80 (vw), 3.73 (w);
wherein (vs)=very strong intensity; (m)=medium intensity; (w)=weak intensity; and (vw)=very weak intensity.

4. A crystalline polymorph of (3R,5S,6E)-7-(4-(4-fluorophenyl)-2,6-diisopropyl-5-(methoxymethyl)pyridin-3-yl)-3,5-dihydroxy-6-heptenoic acid sodium salt which exhibits a characteristic X-ray powder diffraction pattern with characteristic peaks expressed in d-values (Å) at
29.6 (vs), 14.8 (m), 10.0 (vs), 7.5 (vs), 5.61 (vw), 5.14 (vw), 4.77 (vw), 4.32 (w), 3.76 (vw), 3.38 (vw);
wherein (vs)=very strong intensity; (m)=medium intensity; (w)=weak intensity; and (vw)=very weak intensity.

5. A crystalline polymorph of (3R,5S,6E)-7-(4-(4-fluorophenyl)-2,6-diisopropyl-5-(methoxymethyl)pyridin-3-yl)-3,5-dihydroxy-6-heptenoic acid sodium salt which exhibits a characteristic X-ray powder diffraction pattern with characteristic peaks expressed in d-values (Å) at
30.3 (s), 15.3 (w), 10.2 (s), 7.7 (m), 7.5 (w), 5.63 (m), 5.13 (s), 4.75 (m), 4.13 (w), 3.76 (w), 3.65 (vw), 3.49 (vw), 3.31 (vw);
wherein (s)=strong intensity; (m)=medium intensity; (w)=weak intensity; and (vw)=very weak intensity.

6. A crystalline polymorph of (3R,5S,6E)-7-(4-(4-fluorophenyl)-2,6-diisopropyl-5-(methoxymethyl)pyridin-3-yl)-3,5-dihydroxy-6-heptenoic acid sodium salt which exhibits a characteristic X-ray powder diffraction pattern with characteristic peaks expressed in d-values (Å) at
31.4 (s), 15.8 (w), 10.5 (s), 10.1 (w), 9.0 (w), 7.9 (s), 7.5 (w), 7.3 (m), 6.6 (vw), 5.65 (m), 5.19 (s), 4.82 (m), 4.31 (w), 4.18 (vw), 4.08 (vw), 3.85 (vw), 3.76 (vw);
wherein (s)=strong intensity; (m)=medium intensity; (w)=weak intensity; and (vw)=very weak intensity.

7. A crystalline polymorph of (3R,5S,6E)-7-(4-(4-fluorophenyl)-2,6-diisopropyl-5-(methoxymethyl)pyridin-3-yl)-3,5-dihydroxy-6-heptenoic acid sodium salt which exhibits a characteristic X-ray powder diffraction pattern with characteristic peaks expressed in d-values (Å) at
31.2 (s), 15.7 (w), 10.4 (m), 10.0 (vw), 9.0 (vw), 7.8 (m), 7.5 (w), 7.3 (m), 6.6 (vw), 5.64 (m), 5.14 (s), 4.76 (m), 4.28 (vw), 4.13 (vw), 3.78 (vw), 3.66 (vw);
wherein (s)=strong intensity; (m)=medium intensity; (w)=weak intensity; and (vw)=very weak intensity.

8. A crystalline polymorph of (3R,5S,6E)-7-(4-(4-fluorophenyl)-2,6-diisopropyl-5-(methoxymethyl)pyridin-3-yl)-3,5-dihydroxy-6-heptenoic acid sodium salt which exhibits a characteristic X-ray powder diffraction pattern with characteristic peaks expressed in d-values (Å) at
29.9 (s), 15.0 (w), 10.0 (s), 8.9 (vw), 7.5 (s), 7.0 (w), 5.64 (m), 5.15 (m), 4.76 (m), 4.40 (vw), 4.29 (vw), 4.09 (vw), 3.74 (vw), 3.62 (vw);
wherein (s)=strong intensity; (m)=medium intensity; (w)=weak intensity; and (vw)=very weak intensity.

9. A crystalline polymorph of (3R,5S,6E)-7-(4-(4-fluorophenyl)-2,6-diisopropyl-5-(methoxymethyl)pyridin-3-yl)-3,5-dihydroxy-6-heptenoic acid sodium salt which exhibits a characteristic X-ray powder diffraction pattern with characteristic peaks expressed in d-values (Å) at
35.4 (s), 29.9 (m), 25.3 (vs), 17.5 (vw), 12.4 (w), 11.7 (vw), 10.6 (m), 9.4 (w), 8.9 (s), 8.7 (shoulder), 7.5 (m), 7.3 (w), 6.8 (w), 6.2 (m), 5.81 (m), 5.27 (m), 5.13 (w); 4.98 (vw); 4.90 (w); 4.78 (vw); 4.70 (w); 4.63 (vw); 4.56 (w); 4.46 (w); 4.35 (w); 4.16 (s); 4.05 (m); 3.84 (w); 3.76 (m); 3.70 (m); 3.62 (vw); 3.59 (vw); 3.56 (vw); 3.51 (vw); 3.49 (vw); 3.33 (vw); 3.17 (vw); 3.00 (w).
wherein (vs)=very strong intensity; (s)=strong intensity; (m)=medium intensity; (w)=weak intensity; and (vw)=very weak intensity.

10. A crystalline polymorph of (3R,5S,6E)-7-(4-(4-fluorophenyl)-2,6-diisopropyl-5-(methoxymethyl)pyridin-3-yl)-3,5-dihydroxy-6-heptenoic acid sodium salt which exhibits a characteristic X-ray powder diffraction pattern with characteristic peaks expressed in d-values (Å) at
35.0 (m); 27.3 (vs); 21.0 (vw); 11.7 (vw); 11.1 (w); 10.4 (m); 9.1 (s); 8.6 (s); 7.8 (w); 7.0 (vw); 6.6 (vw); 6.04 (m); 5.28 (m); 5.07 (w); 4.82 (w); 4.72 (m); 4.51 (vw); 4.37 (vw); 4.27 (m); 4.11 (m); 3.81 (s); 3.64 (w); 3.57 (vw); 3.51 (vw); 3.43 (vw); 3.36 (vw); 3.25 (w); 2.69 (w)
wherein (vs)=very strong intensity; (s)=strong intensity; (m)=medium intensity; (w)=weak intensity; and (vw)=very weak intensity.

11. A process for the preparation of a crystalline polymorph according to claim 1 wherein the amorphous form or a crystalline polymorph according to claim 9 is placed in an atmosphere with a relative humidity between 30 and 70%.

12. A process for the preparation of a crystalline polymorph according to claim 3 wherein a crystalline polymorph according to claim 1 or 2 is dried, preferably at ambient temperature, and subsequently treated at a temperature between 70 and 150° C.

13. A process for the preparation of a crystalline polymorph according to claim 4 wherein a crystalline polymorph according to claim 1, 2 or 9 is placed in an atmosphere with a relative humidity between 70 and 100%.

14. A pharmaceutical composition comprising an effective amount of a crystalline polymorphic form according to any of claims 1 to 10, and a pharmaceutically acceptable carrier.
